(19) United States
(12) Patent Application Publication
(10) Pub. No.: US 2017/0095216 A1
(43) Pub. Date: Apr. 6, 2017

(54) BIOWATCH

(71) Applicant: Mark Laty, Miami, FL (US)

(72) Inventor: Mark Laty, Miami, FL (US)

(21) Appl. No.: 15/232,080

(22) Filed: Aug. 9, 2016

Related U.S. Application Data
(60) Provisional application No. 62/236,677, filed on Oct. 2, 2015.

(51) Int. Cl. A61B 5/00; A61B 5/145; H04L 12/26; A61B 5/024; H04W 4/02; H04B 1/3827; A61B 5/1455; A61B 5/021

(52) U.S. Cl. CPC A61B 5/746; A61B 5/0002; A61B 5/14551; A61B 5/14532; A61B 5/0008; A61B 5/02141; A61B 5/02438; A61B 5/681; H04W 4/025; H04B 1/385; H04L 43/16

(57) ABSTRACT

A wrist-worn biowatch providing various health monitoring functions such as blood glucose level monitoring, blood pressure detection, pulse monitoring, heart stop detection, oxygen saturation saturation monitoring, and Ketoacidosis detection, wherein the biowatch is actively monitoring the wellness data of its wearer, and adapted to alert the user and medical professionals if such wellness data veers outside normal ranges or acceptable trends.

### CROSS-REFERENCE TO RELATED APPLICATION

This application claims the benefit of priority of U.S. provisional application Ser. No. 62/236,677, filed Oct. 2, 2015, the contents of which are herein incorporated by reference.

### BACKGROUND OF THE INVENTION

The present invention relates to health monitoring devices and, more particularly, to a biowatch providing various health monitoring functions such as blood glucose level monitoring, blood pressure detection, pulse monitoring, heart stop detection, oxygen saturation monitoring, Ketoacidosis detection and location positioning.

For many people, actively monitoring certain vital signs and wellness data can mean the difference between life and death. Many forms of such health monitoring are, however, invasive, requiring the drawing of blood to measure blood glucose level, for example. Likewise, such forms of health monitoring typically require the user to actively seek out and set aside time for the monitoring. Human nature being what it is, many individuals who should be actively monitoring certain vital signs and wellness data do not, even though their life may depend on it.

As can be seen, there is a need for a wrist-worn biowatch providing various health monitoring functions such as blood glucose level monitoring, blood pressure detection, pulse monitoring, heart stop detection, oxygen saturation monitoring, and Ketoacidosis detection, wherein the biowatch is actively monitoring the wellness data of its wearer, and adapted to alert the user and medical professionals if such wellness data veers outside normal ranges or acceptable trends.

### SUMMARY OF THE INVENTION

In one aspect of the present invention, a wrist-worn biowatch including a body defined by a distal surface and an opposing proximal surface; a wrist band connected to the body; a band sensor housing provided along the wrist band, wherein the wrist band is adapted to move between an open condition and a closed condition wrapped around a wrist of a user so that the proximal surface abuts an anterior portion of the wrist, and wherein the band sensor housing abuts a posterior portion of the wrist; and the proximal surface and the band sensor housing each providing at least one wellness sensor adapted to measure wellness data.

In another aspect of the present invention, the wrist-worn biowatch includes a body defined by a distal surface and an opposing proximal surface; a wrist band connected to the body; an audio device; a global positioning system; a band sensor housing provided along the wrist band, wherein the wrist band is adapted to move between an open condition and a closed condition wrapped around a wrist of a user so that the proximal surface abuts an anterior portion of the wrist, and wherein the band sensor housing abuts a posterior portion of the wrist; a pulse sensor disposed along the band sensor housing to abut a radial artery of the posterior portion of the wrist; the proximal surface and the band sensor housing each providing at least one wellness sensor adapted to measure wellness data; a laser coupled to one or more of the at least one wellness sensor; a microprocessor coupled to the pulse sensor and the at least one wellness sensor, wherein the microprocessor is configured to send an alarm when wellness data measured by the at least one wellness sensor exceeds an associated predetermined threshold stored in the microprocessor, wherein the alarm includes an electronically exportable message and an audio output from the audio device, and wherein the electronically exportable includes a current global position as determined by the global positioning system, and wherein the at least one wellness sensor includes a Ketone sensor and wherein the at least one wellness sensor is selected from the group consisting of a blood glucose sensor, a blood pressure sensor, and an oxygen saturation sensor. These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a perspective view of an exemplary embodiment of the present invention, shown in use demonstrating a display mode;

FIG. 3 is a section view of an exemplary embodiment of the present invention, taken along line 3-3 of FIG. 1; and

FIG. 4 is a schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a wrist-worn biowatch providing various health monitoring functions such as blood glucose level monitoring, blood pressure detection, pulse monitoring, heart stop detection, oxygen saturation monitoring, and Ketoacidosis detection, wherein the biowatch is actively monitoring the wellness data of its wearer, and adapted to alert the user and medical professionals if such wellness data veers outside normal ranges or acceptable trends.

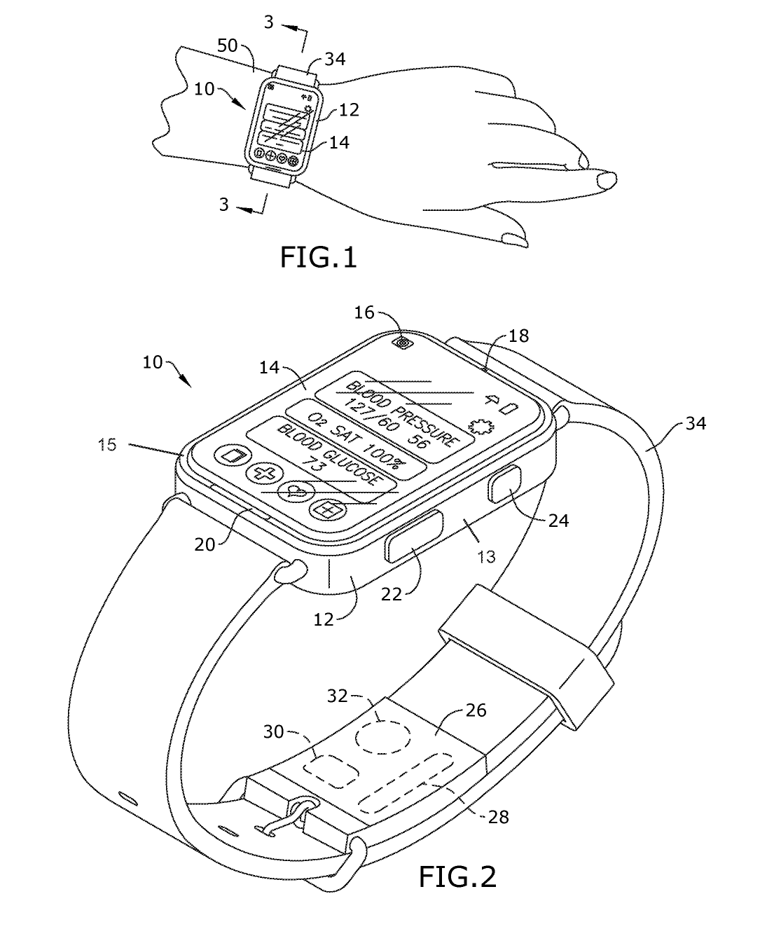

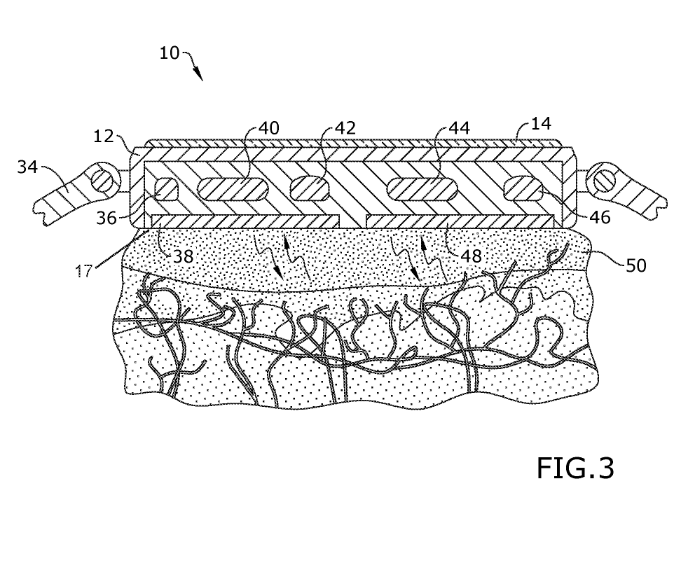

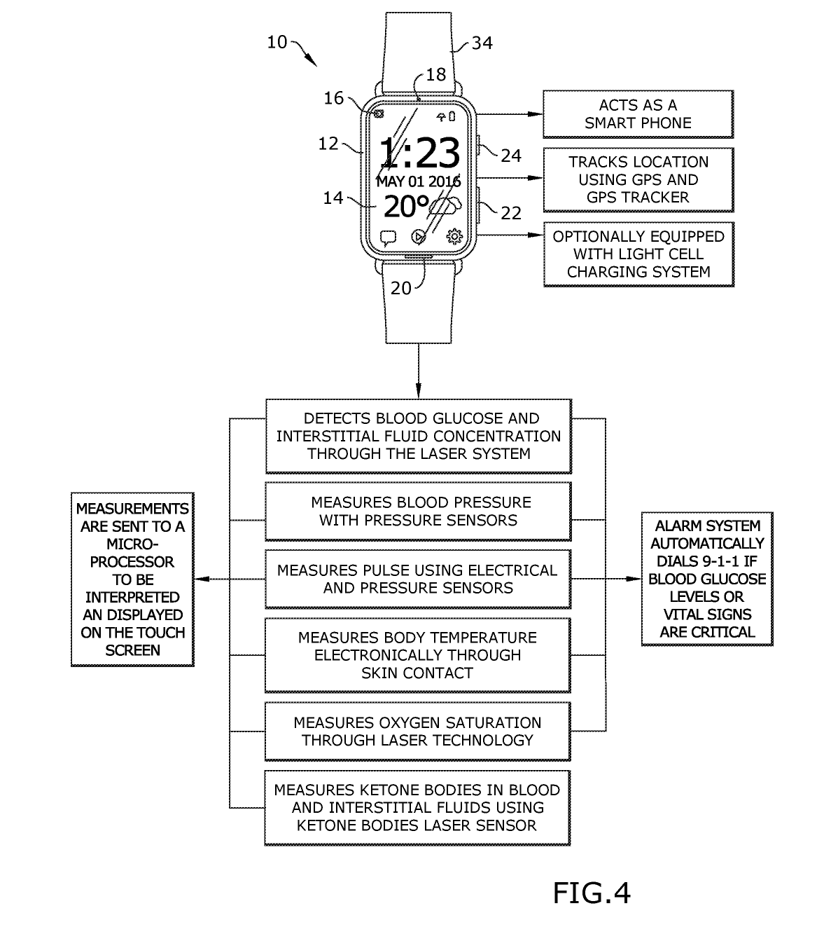

Referring to FIGS. 1 through 4, the present invention may include a biowatch 10 communicably coupled with a microprocessor. The biowatch 10 provides a body 12 connected to a watch band 34 dimensioned and adapted to be movable between an open condition and a closed condition securing the biowatch 10 around the wrist 50 of a user. The body 12 provide a distal surface 15 providing a touchscreen 14 accessible to the other hand of the user when the biowatch 10 is wrist worn, for example when the wrist band 34 is in the closed condition, as illustrated in FIG. 2. Opposite the distal surface 15, interconnected by a plurality of sidewalls 13, is a proximal surface 17 that contacts the anterior portion of the user's wrist 50, as illustrated in FIG. 3. The biowatch 10 may include a GPS/camera 16, a microphone 18, an audio output device 20 such as a speaker, and other convention smart phone technologies. The biowatch 10 may include a plurality of wellness sensors coupled to the microprocessor for measuring wellness data. Wellness data may include blood glucose level, blood pressure level, pulse rate, heart activity, oxygen saturation level, Ketoacidosis levels and the like. The microprocessor may provide an alarm system adapted to analyze the wellness data so that, in one embodiment, the microprocessor generates alarms if the value of any of the wellness data exceeds predetermined thresholds, wherein said thresholds are likely established by medical professionals. In such a case, microprocessor may generate exportable messages, for example phone messages, email messages and SMS messages sent to user-specified addresses, such as 911. Biowatch 10 may be adapted to send a loud alarm through the audio device 20 to its wears if any of the above wellness data is above the predetermined thresholds, automatically calling 911 if the value continues to be alarmingly without interference. The alarm system may be coupled with the GPS so as the above mentioned alarm message (911 call, SMS or email) includes the user's current location.

In another embodiment, the microprocessor may be adapted to represent any of the wellness data on the user interface/touchscreen 14 for the user to toggle to via a change to screen button 24, and toggle back to conventional smart phone technologies via a change to mobile button 22.

The wellness sensors may be housed in the body 12 while others may be housed a band sensor housing 26 provided by the watch band 34. In the closed condition the band sensor housing 26 contacts the posterior portion of the user's wrist when the wrist band 34 is in the closed condition. To wit, a blood pressure sensor 28 and a pulse sensor 30 may be provided in the sensor housing 26, wherein the pulse sensor 30 is disposed (or a portion of the band sensor housing 26 in communication with the pulse sensor 30) abuts the radial artery of the user's wrist 50, thereby the pulse sensor 30 may be adapted to determine a user's pulse when the biowatch 10 is wrist worn. In certain embodiments, the pulse sensor 30 may be pressure and/or electrical sensors adapted to determine the user's pulse and send alarms based on arrhythmia and/or heart disease thresholds. The blood pressure sensor 28 may be pressure pads adapted to determine the user's blood pressure and send alarms based on hypertension and/or hypotension thresholds. Similarly, the pulse sensor 30 and the blood pressure sensor 28 may be adapted to determine if the user's heart stops for a predetermined length of time and send alarms based on heart attack and/or asystole thresholds. The biowatch 10 may provide a temperature sensor 36 coupled directly or indirectly to the proximal surface for measuring the body temperature of the user.

A blood glucose sensor 48 and Ketone sensor 38 may be disposed along the proximal surface 17 of the body 12, as illustrated in FIG. 3. The blood glucose sensor 48 may provide a non-invasive laser sensor or far infrared sensor 46 coupled with an associated receiver 44 so that the blood glucose sensor 48 is adapted to determine the concentration of glucose in the users' serum and send alarms based on diabetes and/or hypoglycemia thresholds. The Ketone sensor 38 may provide a non-invasive laser sensor or far infrared sensor 42 coupled with an associated receiver 40 so that the Ketone sensor 38 is adapted to detect Ketoacidosis in the user's serum and send alarms based on diabetic-related thresholds. It being understood that the lasers 42, 46 are coupled to the skin of the user when the biowatch 10 is in use, such as when wrist worn.

The GPS 16 may include a GPS tracking system adapted so that if the user/wearer becomes lost, the alarm system will call 911 or home by pressing a button. Likewise, a user may push a button if he or she is in danger.

The plurality of wellness sensors may be coupled to a power source 32, such as a battery or an alternative light cell charging system, provided by the biowatch 10. The biowatch 10 is adapted to accept additive technologies, such as a creative level sensor, that can be coupled with the lasers 42, 46 and receivers 40, 44. Likewise, the biowatch 10 may provide an oxygen saturation sensor coupled with a non-invasive laser 42 or 46 and an associated receiver 40 or 44 so that the oxygen saturation sensor 48 is adapted for measuring the oxygen saturation of the user.

The location of the wellness sensors may be arranged differently than shown in the figures, i.e., some shown in the wrist band 34 may be along the proximal surface 17 and vice versa, as long as they function (for example, are touching or otherwise coupled to the skin of the user) in accordance with the present invention has disclosed herein.

A method of using the present invention may include the following. A user may program the microprocessor to set the thresholds associated with each wellness sensor. Similarly, the user may also program each wellness sensor to obtains measurement at certain predetermined intervals, for example the blood glucose sensor 48 may be programmed to determine the user's blood sugar once, twice or so many times a day. The user then wears the biowatch 22 around their wrist with the wrist band 34 in the closed condition so that the band sensor housing 26 is operable, and then uses the change to screen button 24 to get the touchscreen 14 to display representations of the wellness data, wherein the user may toggle back to conventional smart phone technologies via the change to mobile button 22.

It should be understood, of course, that the foregoing relates to exemplary embodiments of the invention and that modifications may be made without departing from the spirit and scope of the invention as set forth in the following claims.

What is claimed is:

1. A wrist-worn device, comprising:
a body defined by a distal surface and an opposing proximal surface;
a wrist band connected to the body;
a band sensor housing provided along the wrist band,
wherein the wrist band is adapted to move between an open condition and a closed condition wrapped around a wrist of a user so that the proximal surface abuts an anterior portion of the wrist, and wherein the band sensor housing abuts a posterior portion of the wrist; and
the proximal surface and the band sensor housing each providing at least one wellness sensor adapted to measure wellness data.

2. The wrist-worn device of claim 1, wherein the least one wellness sensor includes a pulse sensor disposed along the band sensor housing to abut a radial artery of the posterior portion of the wrist.

3. The wrist-worn device of claim 1, further comprising a microprocessor provided coupled to the at least one wellness sensor, wherein the microprocessor is configured to send an alarm when wellness data measured by the at least one wellness sensor exceeds an associated predetermined threshold stored in the microprocessor.

4. The wrist-worn device of claim 3, wherein the alarm includes an electronically exportable message.

5. The wrist-worn device of claim 4, further comprising an audio device provided by the wrist-worn device, wherein the alarm includes an audio output from the audio device.

6. The wrist-worn device of claim 4, further comprising a global positioning system provided by the wrist-worn device, wherein the electronically exportable includes a current global position as determined by the global positioning system.

7. The wrist-worn device of claim 1, further comprising a laser coupled to one or more of the at least one wellness sensor.

8. The wrist-worn device of claim 1, wherein the at least one wellness sensor includes a Ketone sensor.

9. The wrist-worn device of claim 1, wherein the at least one wellness sensor is selected from the group consisting of a blood glucose sensor, a blood pressure sensor, and an oxygen saturation sensor.

10. A wrist-worn device, comprising:
a body defined by a distal surface and an opposing proximal surface;
a wrist band connected to the body;
an audio device;
a global positioning system;
a band sensor housing provided along the wrist band,
wherein the wrist band is adapted to move between an open condition and a closed condition wrapped around a wrist of a user so that the proximal surface abuts an anterior portion of the wrist, and wherein the band sensor housing abuts a posterior portion of the wrist;
a pulse sensor disposed along the band sensor housing to abut a radial artery of the posterior portion of the wrist;
the proximal surface and the band sensor housing each providing at least one wellness sensor adapted to measure wellness data;
a laser coupled to one or more of the at least one wellness sensor;
a microprocessor coupled to the pulse sensor and the at least one wellness sensor, wherein the microprocessor is configured to send an alarm when wellness data measured by the at least one wellness sensor exceeds an associated predetermined threshold stored in the microprocessor, wherein the alarm includes an electronically exportable message and an audio output from the audio device, and wherein the electronically exportable includes a current global position as determined by the global positioning system, and
wherein the at least one wellness sensor includes a Ketone sensor and wherein the at least one wellness sensor is selected from the group consisting of a blood glucose sensor, a blood pressure sensor, and an oxygen saturation sensor.
